(19) United States
(12) Patent Application Publication
(10) Pub. No.: US 2015/0112095 A1
(43) Pub. Date: Apr. 23, 2015

(54) PARTIALLY FLUORINATED ALCOHOLS AND DERIVATIVES

(71) Applicant: E I DU PONT DE NEMOURS AND COMPANY, Wilmington (DE)

(72) Inventor: ROMAN B. LARICHEV, WILMINGTON, DE (US)

(73) Assignee: E I DU PONT DE NEMOURS AND COMPANY, Wilmington (DE)

(21) Appl. No.: 14/055,943

(22) Filed: Oct. 17, 2013

(51) Int. Cl. C07C 69/675; C07C 41/01; C07C 67/00; C07C 43/13; C07C 59/135; C07C 51/00

(52) U.S. Cl. CPC C07C 69/675; C07C 59/135; C07C 51/00; C07C 67/00; C07C 43/137; C07C 41/01
USPC 560/184; 562/586; 568/677

(57) ABSTRACT

The present invention is drawn to a compound of formula (1):

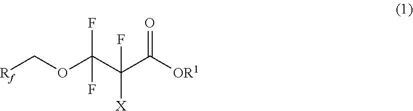

wherein R_{f} is C₁ to C₆ linear or branched fluoroalkyl, X is F or Cl, and R¹ is H, CH₃, or CH₂CH₃. The compound can be used to form partially fluorinated alcohols, derivatives, and polymers. Thus, the present invention provides compounds having lower fluorine content, the process for making lower fluorine containing compounds, and derivatives made from lower fluorine containing alcohols.

### FIELD OF THE INVENTION

The field of invention is related to compounds useful for making partially fluorinated alcohols, partially fluorinated alcohols, and derivatives thereof useful as starting materials for additives in coating compositions and film forming foams, which impart surface effects to substrates coated with such compositions and to film forming foams.

### BACKGROUND OF THE INVENTION

Partially fluorinated alcohol and derivative compounds are commonly prepared from long chain fluorinated iodides or a mixture of long chain fluorinated iodides. These alcohols are expensive and in short supply. Shorter chain fluorinated alcohols would provide a reduction of fluorine in the resulting derivative compounds, which is desirable, if the same or improved performance can be maintained in imparting surface effects to substrates and foams treated therewith. Reduction of fluorine in the fluorinated alcohols would also reduce the cost to produce these materials.

Honda et al., in Macromolecules, 2005, 38, 5699-5705 show that for perfluoroalkyl chains of 8 carbons or greater, orientation of the perfluoroalkyl groups is maintained in a parallel configuration, while reorientation occurs for such chains having 6 carbon atoms or less. Such reorientation decreases surface properties such as receding contact angle. Thus, shorter chain perfluoroalkyls have traditionally not been successful commercially for imparting surface effects to substrates and film forming foams.

Wiley (U.S. Pat. No. 2,988,537) discloses several carbonates, esters, oxalate esters, and amides used as trapping agents to form fluorinated products ranging from ketones with only one fluorinated group, to symmetrical ketones with two fluorinated groups, to fluorinated esters. In the majority of reactions, a mixture of products was obtained. However, the scope of this reaction was not fully developed to include the fluorine efficient intermediates, carboxylic acids, alcohols, and derivatives of the present invention.

It is desirable to have new, more selective chemistry, which provides a more fluorine efficient building block for use as an intermediate, to produce fluorinated polymers providing surface effects to substrates and foams treated therewith. The present invention solves this problem.

### SUMMARY OF INVENTION

The present invention relates to a compound of formula (1):

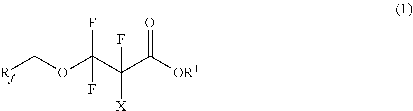

wherein R_{f} is C₁ to C₆ linear or branched fluoroalkyl, X is F or Cl, and R¹ is H, CH₃, or CH₂CH₃.

The present invention also relates to a process to prepare a compound of Formula (1), wherein R_{f} is C₁ to C₆ linear or branched fluoroalkyl and X is F or Cl, comprising a) contacting a alkali metal hydride with a fluorinated alcohol of formula R_{f}CH₂OH to produce a catalyst, b) contacting a symmetrical fluorinated carbonate of formula (R_{f}CH₂O)₂C(O), wherein R_{f} is C₁ to C₆ linear or branched fluoroalkyl, with CF₂═CFX, wherein X is F or Cl, in the presence of the catalyst to generate a reaction mixture, c) hydrolyzing the reaction mixture of step b) to form a compound of Formula (1a),

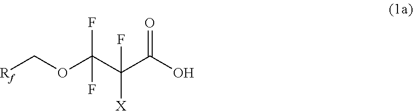

and d) optionally reacting the compound of Formula (1a) with methanol or ethanol to yield a compound of Formula (1).

The invention further relates to a compound of formula (2):

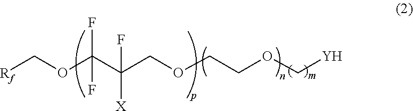

wherein R_{f} is C₁ to C₆ linear or branched fluoroalkyl; X is F or Cl; Y is O or a single bond; p is 0 to 1; m is 0 or 3 to 10; and n is 0 to 30; wherein at least one of p or m is a positive integer; provided that, if n is a positive integer, then p is 1; and provided that, if m is 0 then Y is a single bond, and if m is a positive integer then Y is O.

The invention also relates to a compound of Formula (3):

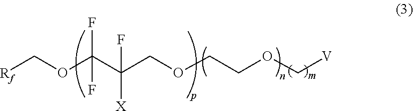

wherein R_{f} is C₁ to C₆ linear or branched fluoroalkyl; X is F or Cl; V is —YC(O)CR²═CH₂, —YC(O)R¹⁹SH, or —YC(O)NHCH₂CH₂OC(O)CR²═CH₂; Y is O or a single bond; R² is independently selected from H or a C₁ to C₄ alkyl group; R¹⁹ is an alkylene of about 1 to about 10 carbon atoms; p is 0 to 1; m is 0 or 3 to 10; and n is 0 to 30; wherein at least one of p or m is a positive integer; provided that, if n is a positive integer, then p is 1; and provided that, if m is 0 then Y is a single bond, and if m is a positive integer then Y is O.

### DETAILED DESCRIPTION OF INVENTION

Hereinafter trademarks are designated by upper case.

The term “derivatives” is used to mean compounds of Formulae (3) to (11) as defined hereinafter.

The term “(meth)acrylates” refers to both acrylates and methacrylates.

The term “(meth)acrylamides” refers to both acrylamides and methacrylamides.

By the term “polyisocyanate” is meant di- and higher isocyanates and the term includes oligomers.

The present invention relates to partially fluorinated carboxylic acids, partially fluorinated alkyl esters, partially fluorinated alcohols, and functionalized derivatives of the partially fluorinated alcohols. The present invention also relates to processes for making the partially fluorinated alcohols through a partially fluorinated aldehyde, through a mixed partially fluorinated carbonate, and through a symmetrical partially fluorinated carbonate.

The present invention relates to a compound of formula (1):

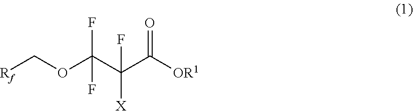

wherein R_{f} is C₁ to C₆ linear or branched fluoroalkyl, X is F or Cl, and R¹ is H, CH₃, or CH₂CH₃. Preferably, R_{f} is C₁ to C₃ linear fluoroalkyl. This compound is useful in the formation of a partially fluorinated alcohol, which can in turn be derivatized and used to produce polymeric materials.

The present invention also relates to a process to prepare a compound of Formula (1), wherein R_{f} is C₁ to C₆ linear or branched fluoroalkyl and X is F or Cl, comprising a) contacting a alkali metal hydride with a fluorinated alcohol of formula R_{f}CH₂OH to produce a catalyst, b) contacting a symmetrical fluorinated carbonate of formula (R_{f}CH₂O)₂C(O), wherein R_{f} is C₁ to C₆ linear or branched fluoroalkyl, with CF₂═CFX, wherein X is F or Cl, in the presence of the catalyst to generate a reaction mixture, c) hydrolyzing the reaction mixture of step b) to form a compound of Formula (1) where R¹ is H designated as formula (1a),

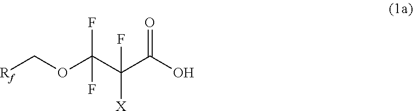

and d) optionally reacting the compound of Formula (1a) with methanol or ethanol to yield a compound of Formula (1). Preferred starting alcohols for step a) include those where R_{f} is C₁ to C₃ linear fluoroalkyl, or mixtures thereof. During step a), an alkali metal hydride of the formula WH_{g}, where W is an alkali metal and g is 1 to 2, is reacted with a fluorinated alcohol of formula R_{f}CH₂OH, wherein R_{f} is C₁ to C₆ linear or branched fluoroalkyl, to produce a catalyst of formula R_{f}CH₂O⁻W⁺.

The process for forming the carboxylic acids of formula (1a) includes producing a symmetrical fluorinated carbonate of formula (7):

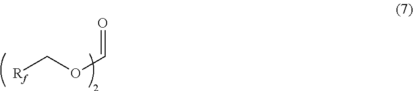

where R_{f} is as defined above, by reacting a fluorinated alcohol, such as 2,2,3,3,3-pentafluoropropanol, with phosgene, diphosgene, or triphosgene in a solvent system, such as pyridine and ether solution. Preferred starting alcohols for forming the symmetrical fluorinated carbonates of Formula (7) have the formula R_{f}CH₂OH, where R_{f} is C₁ to C₆ linear or branched fluoroalkyl, preferably C₁ to C₃ linear fluoroalkyl, or mixtures thereof. Preferably, the same starting alcohol used to form the compound of Formula (7) is also used in step a), resulting in the compound of Formula (1a).

The corresponding symmetrical fluorinated carbonates of formula (7) are then reacted with tetrafluoroethylene or trifluorochloroethylene using catalytic amounts of an alkoxide generated in step a), such as R_{f}CH₂O⁻, to produce compounds of formula (8):

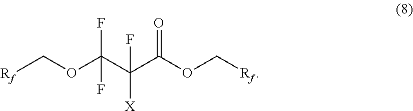

Compounds of formula (8) are then treated with caustic, such as aqueous sodium hydroxide, followed by an acid wash, such as aqueous hydrochloric acid, to produce partially fluorinated carboxylic acids of formula (1a).

Partially fluorinated alkyl esters of Formula (1), where R¹ is CH₃, designated as Formula (1b), or where R¹ is CH₂CH₃, designated as Formula (1c), can be prepared by esterifying the partially fluorinated carboxylic acid, compounds of formula (1a), with an alcohol such as, for example, methanol or ethanol:

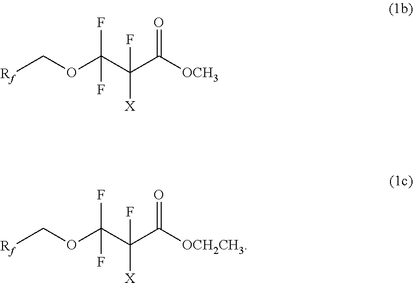

In another embodiment, partially fluorinated alkyl esters of Formula (1), where R¹ is CH₃ (Formula 1b) or CH₂CH₃ (Formula 1c), can be prepared by the insertion of tetrafluoroethylene or trifluorochloroethylene into a mixed partially fluorinated carbonate of Formula (9):

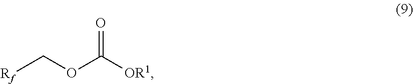

wherein R_{f} is C₁ to C₆ linear or branched fluoroalkyl and R¹ is CH₃ or CH₂CH₃, using catalytic amounts of an alkoxide such as R_{f}CH₂O⁻.

The partially fluorinated alkyl esters of Formula (1) can be reduced to form partially fluorinated alcohols useful in the invention.

Compounds of formula (9) can be prepared by reacting a fluorinated alcohol of formula R_{f}CH₂OH with an alkyl chloroformate and pyridine, followed by an acid wash and isolation. Preferred starting alcohols for forming the mixed partially fluorinated carbonate of Formula (9) have the formula R_{f}CH₂OH, where R_{f} is C₁ to C₆ linear or branched fluoroalkyl, preferably C₁ to C₃ linear fluoroalkyl, or mixtures thereof. The alkyl chloroformate is preferably C₁ to C₆ alkyl chloroformate, and more preferably methyl chloroformate.

In another embodiment, a partially fluorinated aldehyde of formula (10)

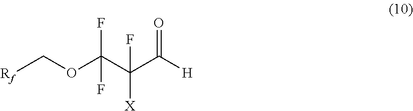

wherein R_{f} is C₁ to C₆ linear or branched fluoroalkyl, can be formed as a useful intermediate for producing a partially fluorinated alcohol. In this embodiment, the process for producing the compound of formula (10) comprises a) contacting an alkali metal hydride with a fluorinated alcohol of formula R_{f}CH₂OH to produce a catalyst, b) contacting R_{f}CH₂OH with formic acid to produce a partially fluorinated formate of Formula (11):

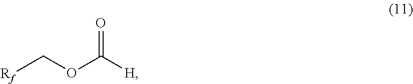

c) contacting the partially fluorinated formate with CF₂═CFX in the presence of the catalyst to form a partially fluorinated aldehyde. Preferably, R_{f} is linear C₁ to C₃ fluoroalkyl, or mixtures thereof.

Compounds of formula (11) can be prepared by reacting fluorinated alcohol of formula R_{f}CH₂OH with formic acid under reflux conditions. Solvents known to those skilled in the art may be also be used throughout the process. In a preferred embodiment, the partially fluorinated formate is formed without the use of solvent.

The invention further relates to a compound of formula (2):

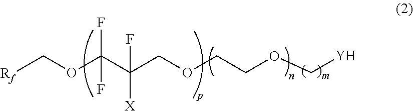

wherein R_{f} is C₁ to C₆ linear or branched fluoroalkyl; X is F or Cl; Y is O or a single bond; p is 0 to 1; m is 0 or 3 to 10; and n is 0 to 30; wherein at least one of p or m is a positive integer; provided that, if n is a positive integer, then p is 1; and provided that, if m is 0 then Y is a single bond, and if m is a positive integer then Y is O. Preferred fluorinated alcohols are those where R_{f} is C₁ to C₃ linear fluoroalkyl. Most preferably, the compound is selected from the formula (2) such that (1) p is 1, m is 0 or 3 to 8, and n is 0; (2) p is 0, m is 3 to 8, and n is 0; or (3) p is 1, m is 0, and n is 1 to 12.

In one embodiment, the partially fluorinated alcohols of formula (2) are made by a process comprising comprising

a) contacting an alkali metal hydride of formula WH_{g}, where W is an alkali metal and g is 1 to 2, with a fluorinated alcohol of formula R_{f}CH₂OH, wherein R_{f} is C₁ to C₆ linear or branched fluoroalkyl, to produce a catalyst of formula R_{f}CH₂O⁻W⁺,

b) contacting R_{f}CH₂OH with (i) formic acid to produce a partially fluorinated formate, (ii) alkyl chloroformate to produce a mixed partially fluorinated carbonate, or (iii) phosgene, diphosgene, or triphosgene to produce a symmetrical partially fluorinated carbonate,

c) contacting the partially fluorinated formate of step (b)(i) or partially fluorinated carbonate of step (b) (ii) or (b) (iii) with CF₂═CFX, wherein X is F or

Cl, in the presence of the catalyst of formula R_{f}CH₂O⁻W⁺ to yield, respectively, a partially fluorinated aldehyde (i), partially fluorinated ester (ii), or partially fluorinated carboxylic acid (iii),

d) contacting the partially fluorinated carboxylic acid of step (c) (iii) with an organic alcohol to form a partially fluorinated ester,

e) contacting (i) the partially fluorinated aldehyde of step (c) (i), (ii) the partially fluorinated ester of step (c) (ii), or (iii) the partially fluorinated ester of step (d) with a reducing agent to form the compound of formula (2) where p is 1 and n=m=0, designated as formula (2a)

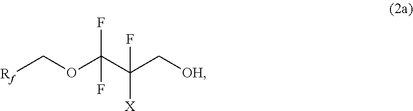

and

f) optionally contacting the compound of formula (2a) with an alcohol of the formula J(CH₂)ₜOH in the presence of a base, where J is a halogen and t is 3-10, or ethylene oxide in the presence of a catalyst, to form the compound of formula (2).

In the above process, R_{f} is preferably linear C₁ to C₃ fluoroalkyl, or mixtures thereof. The alkali metal hydride of step a) can be any alkali metal hydride conventionally used in the art, but is preferably selected from the group consisting of NaH, KH, and CaH₂; and the reducing agent of step e) can be any reducing agent conventionally used in the art but is preferably selected from the group consisting of LiAlH₄ and NaBH₄. Solvents known to those skilled in the art may be also be used throughout the process, including but not limited to ether.

Preferably, the same starting alcohol used to form the partially fluorinated formate of step (b)(i) or partially fluorinated carbonate of step (b) (ii) or (b) (iii) is also used in step a) to form the catalyst of formula R_{f}CH₂O⁻W⁺.

The formation of the partially fluorinated formate of step (b)(i) or partially fluorinated carbonate of step (b) (ii) or (b) (iii), and reduction of the partially fluorinated aldehyde of step (c) (i), the partially fluorinated ester of step (c) (ii), or the partially fluorinated ester of step (d) can be performed by conventional methods known to one of skill in the art. The intermediate compounds may be isolated prior to reaction. Preferably, steps c) and e) are performed as a semi-continuous process where the partially fluorinated aldehyde of step (c) (i) or partially fluorinated ester of step (c) (ii) is not isolated prior to performing step e). In another preferred embodiment, the partially fluorinated formate of step (b) (ii) is formed without the use of organic solvent. In another preferred embodiment, steps (c) (i), (c) (ii), (e) (i), and (e) (ii) take place at a temperature at or below −20° C., most preferably from −20 to −40° C.

Fluorinated alcohols of formula (2a) or R_{f}CH₂OH may be extended to further improve the fluorine efficiency of the molecules. These extended partially fluorinated alcohols are represented by Formulae (2) where p is 1 and m is a positive integer designated as formula (2b); Formulae (2) where p is 0 and m is positive integer designated as formula (2c); and Formula (2) where p is 1 and n is a positive integer designated as formula (2d):

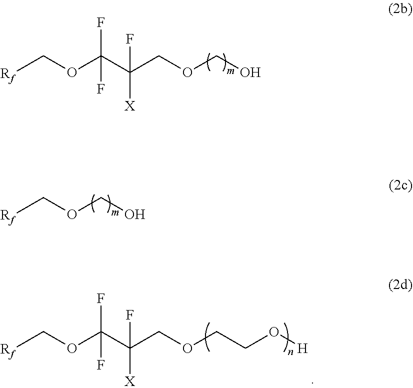

The compounds of Formula (2b) can be formed by the reaction of the partially fluorinated alcohol of Formula (2a) with an alcohol of the formula J(CH₂)ₜOH, where J is a halogen and t is 3 to 10, in the presence of a base. Similarly, the compounds of Formula (2c) can be formed by contacting an alcohol of the formula R_{f}CH₂OH, wherein R_{f} is C₁ to C₆ linear or branched fluoroalkyl, more preferably C₁ to C₃ linear fluoroalkyl, with an alcohol of formula J(CH₂)ₜOH, where J is a halogen and t is 3-10, in the presence of a base. In the compounds of Formulae (2b), m is preferably 3 to 8 and more preferably 3 to 6. In the compounds of Formula (2c), m is preferably 3 to 10, and more preferably 3 to 8. The base used to form the alcohol of formula (2b) or (2c) can be any base conventionally used in the art, but is preferably selected from the group consisting of NaH, KOH, NaOH, Na₂CO₃, and Cs₂CO₃.

The compounds of Formula (2d) can be prepared by reacting partially fluorinated alcohols of Formula (2a) with ethylene oxide in the presence of a catalyst. In the compounds of Formula (2d), n is 1 to 30 and preferably 1 to 12. The catalyst used to form the alcohol of formula (2d) can be any catalyst conventionally used in the art, but is preferably selected from the group consisting of NaH, KOH, NaOH, Cs₂CO₃, and a boron-based catalyst. The term “boron-based catalyst” is hereby defined as a mixture of trialkyl borate B(OR²⁰)₃ and a halide source LE, wherein R²⁰ is a linear, branched, cyclic, or aromatic hydrocarbyl group, optionally substituted, having from 1 to 30 carbon atoms; L is a cation of the alkali metals Na+, K+, Li+ or a cation of an alkyl tertiary amine or alkyl tertiary phosphorus; and E is fluoride, bromide, or iodide. Trialkyl borates are typically prepared in situ by reacting boric acid or sodium borohydride with the alcohol to be ethoxylated. The base compounds, as well as the starting materials for borate synthesis, are readily available from Sigma Aldrich, St. Louis, Mo. The borate/halide catalyst system is described in detail in U.S. Pat. No. 8,067,329, herein incorporated by reference.

It should be understood by one skilled in the art that mixtures of these reactions are also included, where both m and n are positive integers. For example, the ethoxylation of an alcohol of Formulae (2b) or (2c) would form extended partially fluorinated alcohols with further improved fluorine efficiency.

The invention also relates to a compound of Formula (3):

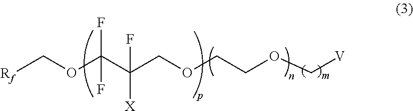

wherein R_{f} is C₁ to C₆ linear or branched fluoroalkyll X is F or Cl; V is —YC(O)CR²═CH₂, —YC(O)R¹⁹SH, or —YC(O)NHCH₂CH₂OC(O)CR²═CH₂; Y is O or a single bond; R² is independently selected from H or a C₁ to C₄ alkyl group; R¹⁹ is an alkylene of about 1 to about 10 carbon atoms; p is 0 to 1; m is 0 or 3 to 10; and n is 0 to 30; wherein at least one of p or m is a positive integer; provided that, if n is a positive integer, then p is 1; and provided that, if m is 0 then Y is a single bond, and if m is a positive integer then Y is O. Preferably, R_{f} is a linear C₁ to C₃ perfluoroalkyl.

Where V is —YC(O)CR²═CH₂ or —YC(O)NHCH₂CH₂OC(O)CR²═CH₂, the invention relates to partially fluorinated (meth)acrylate derivatives. The (meth)acrylate intermediates of the present invention, where V is —YC(O)CR²═CH₂, can be prepared from the alcohols of Formula (2) by adding triethylamine and tetrahydrofuran, then reacting with acryloyl chloride or methacryloyl chloride by adding them dropwise in tetrahydrofuran. The solid is removed, typically by filtration, and washed with tetrahydrofuran, and then purified, usually by ether extraction and water-washing, concentrating and drying under vacuum.

Alternatively, the (meth)acrylate derivatives of the present invention, where V is —YC(O)CR²═CH₂, can be prepared from the alcohols of Formula (2) by reacting with acrylic, methacrylic or chloroacrylic acid in the presence of an acid catalyst, such as toluenesulfonic acid, and a solvent, such as hexane, cyclohexane, heptane, octane, or toluene. The organic layer is washed with water, isolated, and then purified, typically by vacuum distillation. Optionally, inhibitors such as 4-methoxyphenol may be added during or after synthesis.

The partially fluorinated urethane (meth)acrylates of the present invention, where V is —YC(O)NHCH₂CH₂OC(O)CR²═CH₂, are prepared from the alcohols of formula (2) by the reaction with corresponding 2-isocyanatoethyl (meth)acrylate in methylene chloride. The solid product is removed, typically by filtration and purified by repeated washing with a mixture of methylene chloride/hexane. Preferably, the product is formed without the use of a solvent.

Where V is —YC(O)R¹⁹SH, the compounds are partially fluorinated mercaptoalkanoate compounds. The mercaptoalkanoate compounds can be prepared from the alcohols of Formula (2) by reacting with mercaptoalkanoic acid in the presence of an acid catalyst, such as toluenesulfonic acid, and a solvent, such as hexane, cyclohexane, heptane, octane, or toluene. The organic layer is washed with water, isolated, and then purified, typically by vacuum distillation.

The partially fluorinated (meth)acrylate compounds of Formula (3) are useful as monomers for producing copolymers, and the partially fluorinated mercaptoalkanoate compounds of Formula (3) are useful as chain transfer agents in polymerization reactions. The resulting polymers are useful for providing surface effects to a variety of substrates such as hard surfaces, textiles and fibrous substrates, and for producing film forming foams.

The polymers comprise the reaction product of: (a) a compound of Formula (3), or a mixture thereof: wherein R_{f} is C₁ to C₆ linear or branched fluoroalkyl; X is F or Cl; V is —YC(O)CR₂═CH₂, —YC(O)R¹⁹SH, or —YC(O)NHCH₂CH₂OC(O)CR²═CH₂; Y is O or a single bond; R² is independently selected from H or a C₁ to C₄ alkyl group; R¹⁹ is an alkylene of about 1 to about 10 carbon atoms; p is 0 to 1; m is 0 or 3 to 10, and n is 0 to 30; wherein at least one of p or m is a positive integer; provided that, if n is a positive integer, then p is 1; and provided that, if m is 0 then Y is a single bond, and if m is a positive integer then Y is O; and (b) at least one ethylenically unsaturated monomer having a functional group selected from a linear or branched hydrocarbon, alcohol, anhydride, ether, ester, formate, carboxylic acid, carbamate, urea, amine, amide, sulfonate, sulfonic acid, sulfonamide, halide, saturated or unsaturated cyclic hydrocarbon, morpholine, pyrrolidine, piperidine, or mixtures thereof.

The ethylenically unsaturated monomer (b) can be any monomer having an ethylenically unsaturated bond with a functional group described above, including but not limited to linear or branched alkyl (meth)acrylates, amino and diamino (meth)acrylates, alkoxylated (meth)acrylates, (meth)acylic acid, vinyl or vinylidene chloride, glycidyl (meth)acrylate, vinyl acetate, hydroxyalkylene (meth)acrylate, urethane or urea (meth)acrylates, (meth)acrylamides including N-methyloyl acrylamide, styrene, alpha-methylstyrene, chloromethyl-substituted styrene, ethylenediol di(meth)acrylate, 2-acrylamido-2-methyl-1-propane sulfonic acid (AMPS), maleic anhydride, and fluorinated (meth)acrylates 2-[methyl[(3,3,4,4,5,5,6,6,7,7,8,8,8-tridecafluorooctyl)sulfonyl]amino]ethyl acrylate having the structure CH₂═CH—COO—C₂H₄—N(CH₃)—SO₂—C₂H₄—C₆F₁₃, 2-[methyl[(3,3,4,4,5,5,6,6,6-nonfluorohexyl)sulfonyl]amino]ethyl acrylate, 2-[methyl[(3,3,4,4,5,5,6,6,7,7,8,8,8-tridecafluorooctyl)sulfonyl]amino]ethyl methacrylate, and 2-[[(3,3,4,4,5,5,6,6,7,7,8,8,8-tridecafluorooctyl)sulfonyl]amino]ethyl methacrylate.

Polymers can be useful for imparting surface effects, wherein compound (a) of Formula (3) is reacted with (b) a monomer selected from Formula (4) or Formula (5), or a mixture thereof:

CH₂═C(R³)COZ(CH₂)_{q}(CHR³)ᵢNR⁴R⁵ Formula (4)

CH₂═C(R³)NR⁶R⁷ Formula (5)

wherein R³ is independently selected from H or CH₃; R⁴ and R⁵ are each independently C₁ to C₄ alkyl, hydroxyethyl, or benzyl; or R⁴ and R⁵ together with the nitrogen atom form a morpholine, pyrrolidine, or piperidine ring; R⁶ and R⁷ are each independently selected from H or C₁ to C₄ alkyl; Z is —O— or —NR⁸— wherein R⁸ is H or C₁-C₄ alkyl; i is 0 to 4, and q is 1 to 4; provided that the nitrogen bonded to R⁴ and R⁵ is from about 0% to 100% salinized, quaternized, or present as amine oxide; and (c) a monomer of Formula (6), or a mixture thereof:

CH₂═CR⁹(R¹⁰)ₑ—COOH Formula (6)

wherein R⁹ is independently selected from H or CH₃; R¹⁰ is CO(CH₂)₅, CO(O)(CH₂)₂, C₆H₄—CHR⁹, or C(O)NR⁹R¹¹, R¹¹ is C₆H₄OCH₂, (CH₂)_{d}, or C(CH₃)₂CH₂; d is 1 to about 10; and e is 0 or 1. Additional monomers may also be copolymerized, selected from those described above for ethylenically unsaturated monomer (b).

In one preferred embodiment, the polymer above employs a compound of Formula (3) such that V is —YC(O)CR²═CH₂ or —YC(O)NHCH₂CH₂OC(O)CR²═CH₂. Preferably, the compounds (a), (b), and (c) are reacted in the following percentages by weight: from about 30% to about 90% of a compound of Formula (3), from about 9% to about 40% of a monomer of Formula (4), and from about 1% to about 30% of a monomer of Formula (6), wherein the sum of the monomer components equals 100%.

The copolymers of this preferred embodiment can be synthesized by any means known to one skilled in the art. The copolymer may be optionally partially or completely salinized or quarternized by conventional techniques known to those skilled in the art, with the degree of salinization or quarternization preferably from about 50% to about 100%. Preferably, the copolymers are synthesized by combining the monomers in a solvent system, such as isopropyl alcohol and methyl isobutyl ketone, heating the mixture to the activation temperature of an initiator, slowly introducing an initiator into the monomer mixture, and allowing the copolymerization to propagate. The polymer mixture is then contacted with an aqueous salinization solution, such as acetic acid solution, and the organic solvents are removed, preferably by distillation. The final product is an aqueous emulsion.

In another preferred embodiment, the polymer above employs a compound of Formula (3) such that V is —YC(O)R¹⁹SH. Preferably, the compounds (a), (b), and (c) are reacted in the following percentages by weight: from about 4% to about 40% of a compound of Formula (3), from about 30% to about 95% of a monomer of Formula (5), and from about 1% to about 30% of a monomer of Formula (6), wherein the sum of the monomer components equals 100%. The polymers of this preferred embodiment can be synthesized by any means known to one skilled in the art. Preferably, the monomer(s) are charged with the partially fluorinated mercaptoalkanoate in a solvent, such as acetonitrile, and purged with nitrogen. The mixture is heated to the activation temperature of an initiator, and water and initiator are charged into the mixture. Additional monomer may be charged at this time as well. The mixture is heated to allow the polymerization to propagate, and the organic solvent is removed, preferably by distillation. The final product is an aqueous emulsion.

Another polymer is useful, having at least one carbamate linkage prepared by: (i) reacting (a) at least one diisocyanate, polyisocyanate, or mixture thereof, having isocyanate groups, and (b) at least one fluorinated compound selected from the formula (2) wherein R_{f} is C1 to C6 linear or branched fluoroalkyl; X is F or Cl; Y is O or a single bond; p is 0 to 1; m is 0 or 3 to 10; and n is 0 to 30, wherein at least one of p or m is a positive integer; provided that, if n is a positive integer, then p is 1; and provided that, if m is 0 then Y is a single bond, and if m is a positive integer then Y is O; and (ii) optionally reacting with (c) water, a linking agent, or a mixture thereof. Preferred embodiments include polymers, where R_{f} is C₁ to C₃ linear fluoroalkyl, said fluorinated compound reacts with about 5 mol % to about 90 mol % of said isocyanate groups, said non-fluorinated compound reacts with about 0.1 mol % to about 60 mol % of said isocyanate groups, the linking agent is a diamine or polyamine, and/or the polymer is in the form of an aqueous dispersion or solution.

The diisocyanate or polyisocyanate reactant used in the formation of the polymer containing at least one carbamate linkage adds to the branched nature of the polymer. Any polyisocyanate having predominately two or more isocyanate groups, or any isocyanate precursor of a polyisocyanate having predominately two or more isocyanate groups, is suitable for use in this invention. For example, hexamethylene diisocyanate homopolymers are suitable for use herein and are commercially available. It is recognized that minor amounts of diisocyanates may remain in products having multiple isocyanate groups. An example of this is a biuret containing residual small amounts of hexamethylene diisocyanate.

Also suitable for use as the polyisocyanate reactant are hydrocarbon diisocyanate-derived isocyanurate trimers. Preferred is DESMODUR N-3300 (a hexamethylene diisocyanate-based isocyanurate available from Bayer Corporation, Pittsburgh, Pa.). Other triisocyanates useful for the purposes of this invention are those obtained by reacting three moles of toluene diisocyanate with 1,1,1-tris-(hydroxymethyl)ethane or 1,1,1-tris (hydroxymethyl)propane. The isocyanurate trimer of toluene diisocyanate and that of 3-isocyanatomethyl-3,4,4-trimethylcyclohexyl isocyanate are other examples of triisocyanates useful for the purposes of this invention, as is methane-tris-(phenylisocyanate). Precursors of polyisocyanate, such as diisocyanate, are also suitable for use in the present invention as substrates for the polyisocyanates. DESMODUR N-3600, DESMODUR Z-4470, and DESMODUR XP 2410, from Bayer Corporation, Pittsburgh, Pa., and bis-(4-isocyanatocylohexyl)methane are also suitable in the invention.

Preferred polyisocyanate reactants are the aliphatic and aromatic polyisocyanates containing biuret structures, or polydimethyl siloxane containing isocyanates. Such polyisocyanates can also contain both aliphatic and aromatic substituents.

Particularly preferred as the polyisocyanate reactant for all the embodiments of the invention herein are hexamethylene diisocyanate homopolymers commercially available, for instance as DESMODUR N-100, DESMODUR N-75 and DESMODUR N-3200 from Bayer Corporation, Pittsburgh, Pa.; 3-isocyanatomethyl-3,4,4-trimethylcyclohexyl isocyanate available, for instance as DESMODUR I (Bayer Corporation); bis-(4-isocyanatocylohexyl)methane available, for instance as DESMODUR W (Bayer Corporation) and diisocyanate trimers of formulae (12a), (12b), (12c) and (12d):

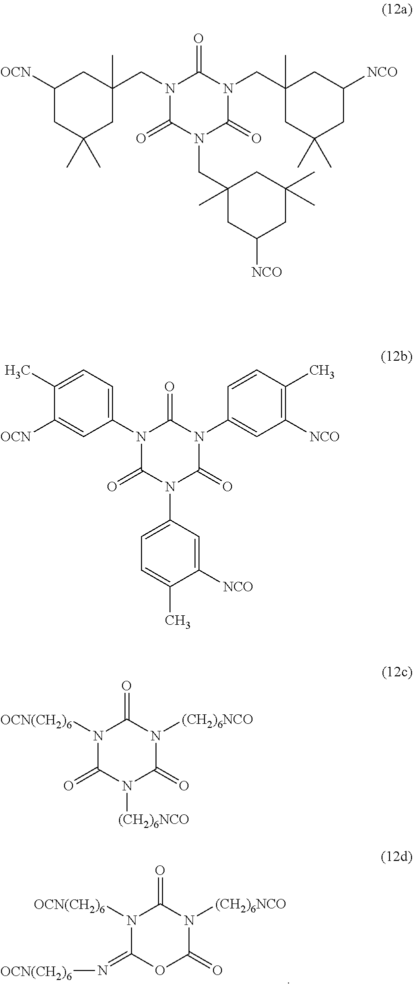

The diisocyanate trimers (12a-d) are available, for instance as DESMODUR Z4470, DESMODUR IL, DESMODUR N-3300, and DESMODUR XP2410, respectively, from Bayer Corporation.

To make the polymers having at least one carbamate linkage of the present invention, a compound of Formula (2) is reacted with a polyisocyanate to produce a fluoropolymer. The fluoropolymer is typically prepared by charging a reaction vessel with the polyisocyanate, the above fluoroalcohol, fluorothiol or fluoroamine, or mixture thereof, and optionally a non-fluorinated organic compound. The order of reagent addition is not critical. The specific weight of the polyisocyanate and other reactants charged is based on their equivalent weights and on the working capacity of the reaction vessel, and is adjusted so that alcohol, thiol or amine, are consumed in the first step. The charge is agitated and temperature adjusted to about 40-70° C. Typically a catalyst, such as a titanium chelate or iron trichloride in an organic solvent, is then added and the temperature is raised to about 80° C.-100° C. Alternatively, the catalyst may be included in the original charge, and the fluorinated alcohol may be added slowly after initial heating. After holding for several hours, additional solvent and water, a linking agent, or a combination thereof, is added, and the mixture allowed to react for several more hours or until all of the isocyanate has been reacted. More water can then be added along with surfactants, if desired, and stirred until thoroughly mixed. Following homogenization, the organic solvent can be removed by evaporation at reduced pressure, and the remaining aqueous solution of the fluoropolymer used as is or subjected to further processing.

Preferably, step (i) further comprises reacting (d) a non-fluorinated organic compound selected from the group consisting of Formula (13)

R¹²—(R¹³)ₖ-QH (13)

wherein R¹² is a C₁-C₁₈ alkyl, a C₁-C₁₈ omega-alkenyl radical or a C₁-C₁₈ omega-alkenyl; k is 0 or 1; Q is —O—, —S—, or —NR¹⁷— in which R¹⁷ is H or alkyl containing 1 to 6 carbon atoms; and R¹³ is selected from the group consisting of

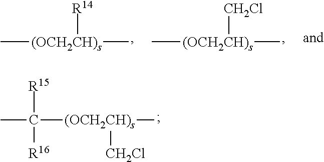

wherein R¹⁴, R¹⁵ and R¹⁶ are each independently H or C₁ to C₆ alkyl, and s is an integer of 1 to 50. Preferably, the compound of formula (13) comprises a hydrophilic water-solvatable material comprising at least one hydroxy-terminated polyether of formula (14):

R¹⁸—O(CH₂CH₂O)ⱼ—(CH₂CH(CH₃)O)ⱼ₁—(CH₂CH₂O)ⱼ₂—H Formula (14)

wherein R¹⁸ is a monovalent hydrocarbon radical containing from about one to about six aliphatic or alicyclic carbon atoms; j is a positive integer, and j1 and j2 are each independently a positive integer or zero; said polyether having a weight average molecular weight up to about 2000.

Subscripts j and j2 are independently an average number of repeating oxyethylene groups, and j1 is an average number of repeating oxypropylene groups, respectively. When j1 and j2 are zero, Formula (14) designates an oxyethylene homopolymer. When j1 is a positive integer and j2 is zero, Formula (14) designates a block or random copolymer of oxyethylene and oxypropylene. When j1 and j2 are positive integers, Formula (14) designates a triblock copolymer designated PEG-PPG-PEG (polyethylene glycol-polypropylene glycol-polyethylene glycol). More preferably, the hydrophilic, water-solvatable components are the commercially available methoxypolyethylene glycols (MPEG's), or mixtures thereof, having an average molecular weight equal to or greater than about 200, and most preferably between 350 and 2000. Also commercially available, and suitable for the preparation of the polyfluoro organic compounds of the present invention, are butoxypolyoxyalkylenes containing equal amounts by weight of oxyethylene and oxypropylene groups (Union Carbide Corp. 50-HB Series UCON Fluids and Lubricants) and having an average molecular weight greater than about 1000.

The non-fluorinated compound of formula R¹²—(R¹³)ₖ-QH is reacted in step (i) with the polyisocyanate and fluorinated compound of formula (2) as described above, prior to the reaction with water, linkage agent, or a mixture thereof. This initial reaction is conducted so that less than 100% of the polyisocyanate groups are reacted. Following the initial reaction, water, linkage agent, or a mixture thereof, is added. The addition of water or linkage agent completely reacts all of the isocyanate groups and eliminates a further purification step that would be needed if other reactants were used at a ratio sufficient to react with 100% of the isocyanate groups. Further, this addition greatly increases the molecular weight of the polymers and assures proper mixing if more than one reactant is used in the first step of the polymer preparation, i.e., if a water solvatable component is added, it is likely that at least one unit will be present in each polymer.

Linking agents useful in forming polymers of the invention organic compounds have two or more Zerewitinoff hydrogen atoms (Zerevitinov, Th., Quantitative Determination of the Active Hydrogen in Organic Compounds, Berichte der Deutschen Chemischen Gesellschaft, 1908, 41, 2233-43). Examples include compounds that have at least two functional groups that are capable of reacting with an isocyanate group. Such functional groups include hydroxyl, amino and thiol groups. Examples of polyfunctional alcohols useful as linking agents include: polyoxyalkylenes having 2, 3 or 4 carbon atoms in the oxyalkylene group and having two or more hydroxyl groups, for instance, polyether diols such as polyethylene glycol, polyethylene glycol-polypropylene glycol copolymers, and polytetramethylene glycol; polyester diols, for instance, the polyester diols derived from polymerization of adipic acid, or other aliphatic diacids, and organic aliphatic diols having 2 to 30 carbon atoms; non-polymeric polyols including alkylene glycols and polyhydroxyalkanes including 1,2-ethanediol, 1,2-propanol diol, 3-chloro-1,2-propanediol, 1,3-propanediol, 1,3-butanediol, 1,4-butanediol, 2,2-dimethyl-1,3-propanediol, 1,2-, 1,5-, and 1,6-hexanediol, 2-ethyl-1,6-hexanediol, 1,8-octanediol, 1,10-decanediol, glycerine, trimethylolethane, trimethylolpropane, 2-ethyl-2-(hydroxymethyl)-1,3-propanediol, 1,2,6-hexanetriol, and pentaerythritol.

Preferred polyfunctional amines useful as linking agents include: amine terminated polyethers such as, for example, JEFFAMINE D400, JEFFAMINE ED, and JEFFAMINE EDR-148, all from Huntsman Chemical Company, Salt Lake City, Utah; aliphatic and cycloaliphatic amines including amino ethyl piperazine, 2-methyl piperazine, 4,4′-diamino-3,3′-dimethyl dicyclohexylmethane, 1,4-diaminocyclohexane, 1,5-diamino-3-methylpentane, isophorone diamine, ethylene diamine, diethylene triamine, triethylene tetraamine, triethylene pentamine, ethanol amine, lysine in any of its stereoisomeric forms and salts thereof, hexane diamine, and hydrazine piperazine; and arylaliphatic amines such as xylylenediamine and a,a,a′,a′-tetramethylxylylenediamine.

Mono- and di-alkanolamines that can be used as linking agents include: monoethanolamine, monopropanolamine, diethanolamine, dipropanolamine, and the like.

The polymers useful for providing surface effects, including those made from ethylenically unsaturated monomers and those from polyisocyanate reactions, are useful in improving the surface effects of various foam formulations, coating compositions and treated substrates. The polymers useful for providing surface effects produce superior surface altering performance, such as, but not limited to wetting, leveling, resistance to blocking, oil repellency, and dirt pickup resistance, of coating compositions while requiring less fluorine, and less fluorinated starting material compared to the prior art compositions. The partially fluorinated polymers may also provide surface tension lowering for applications such as aqueous film forming foams, often used to extinguish hydrocarbon fuel fires. This improved performance results in a reduced cost of raw materials and manufacturing as well as decreases cycle time.

Typical substrates include a wide variety of surfaces on which coating compositions are normally used. These include various construction materials, typically hard surfaced materials. The hard surface substrates include porous and non-porous mineral surfaces, such as glass, stone, masonry, concrete, unglazed tile, brick, porous clay and various other substrates with surface porosity.

Specific examples of such substrates include unglazed concrete, brick, tile, stone including granite, limestone and marble, grout, mortar, statuary, monuments, wood, composite materials such as terrazzo, and wall and ceiling panels including those fabricated with gypsum board. In addition plastics, metals, ceramics, and other hard surfaces are included in the present invention. These are used in the construction of buildings, siding, roads, parking ramps, driveways, floorings, fireplaces, fireplace hearths, counter tops, walls, ceilings, decks, patios, furniture, fixtures, appliances, molded articles, shaped articles, decorative articles, and other items used in interior and exterior applications.

Other substrates include fibrous substrates. Most any fibrous substrate is suitable for treatment by the methods of the present invention. Such substrates include fibers, yarns, fabrics, fabric blends, textiles, carpet, rugs, nonwovens, leather and paper. The term “fiber” includes fibers and yarns, before and after spinning, of a variety of compositions and forms, and includes pigmented fibers and pigmented yarns. By “fabrics” is meant natural or synthetic fabrics, or blends thereof, composed of fibers such as cotton, rayon, silk, wool, polyester, polypropylene, polyolefins, nylon, and aramids such as “NOMEX” and “KEVLAR”. By “fabric blends” is meant fabric made of two or more different fibers. Typically these blends are a combination of at least one natural fiber and at least one synthetic fiber, but also can be a blend of two or more natural fibers and/or of two or more synthetic fibers.

It is desirable to have new, more selective chemistry, which provides a more fluorine efficient building block for use as an intermediate, to produce fluorinated polymers providing surface effects to substrates and foams treated therewith. The present invention solves this problem. Included are lower fluorine containing starting alcohols, the process for making lower fluorine containing starting alcohols, derivatives made from the lower fluorine containing starting alcohols, and polymers made from the lower fluorine containing starting alcohols and derivatives. The derivatives and polymers of the present invention serve to improve surfactant performance, such as lower the surface tension of a coating composition or foam composition, while using less fluorine. They can also impart surface effects, such as water and oil repellency, to coated surfaces.

### Materials and Test Methods

### Materials

Unless otherwise noted, all of the chemicals used herein are commercially available from Sigma Aldrich, St. Louis, Mo.

2,2,3,3,3-pentafluoropropanol is commercially available from Oakwood Products Inc.
2,2,3,3,4,4,4-heptafluorobutanol is commercially available from Oakwood Products Inc.
Triphosgene is commercially available from TCI America.
Phosphorus Pentoxide is commercially available from Filo Chemical or from Changzhou Qishuyan Fine Chemical CO., LTD.

### EXAMPLES

### Example 1

Example 1 illustrates the preparation of bis(2,2,3,3,3-pentafluoropropyl) carbonate and the subsequent preparation of 2,2,3,3-tetrafluoro-3-(2,2,3,3,3-pentafluoropropoxy)propanoic acid. Triphosgene (24.5 g, 82.5 mmol) and anhydrous diethyl ether (˜400 mL) were added to a 1-L 4-neck flask. The mixture was cooled to 0° C., and 2,2,3,3,3-pentafluoropropanol (75 g, 0.50 mol) was added. The mixture was stirred for 30 minutes. Pyridine (40.0 g, 0.51 mol) was then slowly added to the mixture via addition funnel. The resultant mixture was then gently refluxed for 1 hour. The solution was filtered to remove white solids and washed with dilute hydrochloric acid solution. The solution was then vacuum distilled to remove ether resulting in bis(2,2,3,3,3-pentafluoropropyl) carbonate (CF₃CF₂CH₂O)₂CO (71 g, 88% yield).

A catalyst was first prepared by slow addition of 2,2,3,3,3-pentafluoropropan-1-ol (15.0 g, 100 mmol) to a suspension of sodium hydride (60% in mineral oil, 6.0 g, 150 mmol) in anhydrous tetrahydrofuran (300 mL) in a 500-mL flask. The resultant mixture was stirred for 15 minutes, transferred into a Hastelloy vessel (1 L), and cooled to −20° C. The bis(2,2,3,3,3-pentafluoropropyl) carbonate, (CF₃CF₂CH₂O)₂CO, (115 g, 353 mmol) was then added to the vessel. The vessel was pressurized with tetrafluoroethylene (60 g, 600 mmol), and the contents were warmed to room temperature and agitated for 6 hours. The reaction mixture was then treated with a solution of NaOH (15 g, 375 mmol) in water (100 mL). Tetrahydrofuran and water were removed to vacuum, and the resultant solids were dissolved by addition of 3.0M hydrochloric acid (400 mL). The organic phase was separated to yield 2,2,3,3-tetrafluoro-3-(2,2,3,3,3-pentafluoropropoxy)propanoic acid C₂F₅CH₂OCF₂CF₂C(O)OH (60 g, 58% yield).

### Example 2

Example 2 is a repeat of Example 1, with a longer fluorinated chain on the alcohol. Triphosgene (24.5 g, 82.6 mmol) and anhydrous diethyl ether (˜400 mL) were added to a 1-L 4-neck flask. The mixture was cooled to 0° C. and 2,2,3,3,4,4,4-heptafluorobutanol (100 g, 0.50 mol) was added and the mixture was stirred for 30 minutes. Pyridine (40.0 g, 0.51 mol) was then slowly added to the mixture via addition funnel. The resultant mixture was then gently refluxed for 1 hour. The solution was filtered to remove white solids and washed with dilute hydrochloric acid solution. The solution was then vacuum distilled to remove ether resulting in bis(2,2,3,3,4,4,4-heptafluorobutyl)carbonate (CF₃CF₂CF₂CH₂O)₂CO (82 g, 80% yield).

A catalyst was first prepared by slow addition of 2,2,3,3,4,4,4-heptafluorobutan-1-ol (15.0 g, 75 mmol) to a suspension of sodium hydride (60% in mineral oil, 4.3 g, 108 mmol) in anhydrous tetrahydrofuran (130 mL) in a 500-mL flask. The resultant mixture was stirred for 15 minutes, transferred into a Hastelloy vessel (400 mL), and cooled to −20° C. Then bis(2,2,3,3,4,4,4-heptafluorobutyl)carbonate (64 g, 150 mmol) was added and the vessel was pressurized with tetrafluoroethylene (30 g, 300 mmol). The vessel was allowed to warm to the ambient temperature and agitated for 6 hours. Then the reaction mixture was treated with a solution of NaOH (8 g, 200 mmol) in water (50 mL). Tetrahydrofuran and water were removed to vacuum, and the resultant solids were dissolved in hydrochloric acid solution (300 mL at 2.0M). The organic phase was separated to give 2,2,3,3-tetrafluoro-3-(2,2,3,3,4,4,4-heptafluorobutoxy)propanoic acid C₃F₇CH₂OCF₂CF₂C(O)OH (36 g, 70% yield).

### Example 3

Example 3 illustrates the preparation of an ethyl ester of Example 1. 2,2,3,3-tetrafluoro-3-(2,2,3,3,3-pentafluoropropoxy)propanoic acid (65 g, 220 mmol), ethanol (50 mL, excess), and concentrated sulfuric acid (50 g) were added to a 250 ml, round bottom flask. The resultant mixture was refluxed for three hours under atmosphere of nitrogen. The product mixture was slowly added to water (400 mL), the organic layer was separated, washed with water (2×50 mL), and dried over magnesium sulfate to yield ethyl 2,2,3,3-tetrafluoro-3-(2,2,3,3,3-pentafluoropropoxy)propanoate C₂F₅CH₂OCF₂CF₂C(O)OCH₂CH₃ (70 g, 98% yield).

### Example 4

Example 4 illustrates the preparation an ethyl ester of Example 2. 2,2,3,3-tetrafluoro-3-(2,2,3,3,4,4,4-heptafluorobutoxy)propanoic acid (34 g, 99 mmol), ethanol (30 mL), and concentrated sulfuric acid (20 g) were added to a 250 mL round bottom flask. The resultant mixture was refluxed for three hours under atmosphere of nitrogen. The product mixture was slowly added to water (300 mL), the organic layer was separated, washed with water (2×50 mL), and dried over magnesium sulfate to yield ethyl 2,2,3,3-tetrafluoro-3-(2,2,3,3,4,4,4-heptafluorobutoxy)propanoate C₃F₇CH₂OCF₂CF₂C(O)OCH₂CH₃ (35 g, 95% yield).

### Example 5

Example 5 illustrates the preparation of 2,2,3,3-tetrafluoro-3-(2,2,3,3,3-pentafluoropropoxy)propan-1-ol from the ethyl ester in Example 3. Lithium aluminum hydride (5.2 g, 137 mmol) and anhydrous ether (100 mL) were added to a 250-mL round bottom flask, and the mixture was cooled to 5° C. Ethyl 2,2,3,3-tetrafluoro-3-(2,2,3,3,3-pentafluoropropoxy)propanoate (77 g, 240 mmol) was added dropwise, keeping the temperature between 5 and 20° C. The mixture was then washed with diluted hydrochloric acid solution, and the organic phase was separated. The organic phase was purified via distillation to yield 2,2,3,3-tetrafluoro-3-(2,2,3,3,3-pentafluoropropoxy)propan-1-ol C₂F₅CH₂OCF₂CF₂CH₂OH (57 g, 85% yield).

### Example 6

Example 6 illustrates the preparation of 2,2,3,3-tetrafluoro-3-(2,2,3,3,4,4,4-heptafluorobutoxy)propan-1-ol from the ethyl ester in Example 4. Lithium aluminum hydride (2.2 g, 58 mmol) and anhydrous ether (50 mL) were added to a 250-mL round bottom flask. The mixture was cooled to 5° C. and stirred. ethyl 2,2,3,3-tetrafluoro-3-(2,2,3,3,4,4,4-heptafluorobutoxy)propanoate (37 g, 99 mmol) was added drop wise to keep the temperature between 5 and 20° C. The mixture was then washed with diluted hydrochloric acid solution, and the organic phase was separated. The organic phase was purified via distillation to yield 2,2,3,3-tetrafluoro-3-(2,2,3,3,4,4,4-heptafluorobutoxy)propan-1-ol C₃F₇CH₂OCF₂CF₂CH₂OH (24 g, 74% yield).

### Example 7

Example 7 illustrates the preparation of 2,2,3,3,4,4,4-heptafluorobutyl methyl carbonate from 2,2,3,3,4,4,4-heptafluorobutan-1-ol, followed by the subsequent preparation of 2-chloro-2,3,3-trifluoro-3-(2,2,3,3,4,4,4-heptafluorobutoxy)propan-1-ol. Anhydrous ether (180 mL), methyl chloroformate (50 g, 530 mmol), and 2,2,3,3,4,4,4-heptafluorobutan-1-ol (100 g, 500 mmol) were added to a 500-mL round bottom flask. The mixture was stirred at ambient temperature for 30 minutes. Then pyridine (42 g, 530 mmol) was added dropwise with stirring, keeping the temperature between 5 and 15° C. More methyl chloroformate (18.0 g, 190 mmol) was added to the mixture followed by the addition of more pyridine (15 g, 190 mmol) at 5-15° C. The reaction mixture was washed with a 2M solution of hydrochloric acid in water (200 mL) and the organic phase was collected. The organic phase was purified via distillation to yield 2,2,3,3,4,4,4-heptafluorobutyl methyl carbonate C₃F₇CH₂OC(O)OCH₃ (91 g, 71% yield).

Sodium hydride (60% in mineral oil, 1.4 g, 35 mmol) and anhydrous tetrahydrofuran (80 mL) were added to a 250-mL flask under nitrogen atmosphere. Then 2,2,3,3,4,4,4-heptafluorobutan-1-ol (5.0 g, 25 mmol) was slowly added. The mixture was cooled to −10° C., and 2,2,3,3,4,4,4-heptafluorobutyl methyl carbonate (25.0 g, 97 mmol) was added followed by slow addition of chlorotrifluoroethylene (11.5 g, 99 mmol) by bubbling through the solution. The resultant mixture was stirred at 0° C. for 30 minutes. Next, lithium aluminum hydride (2.0 g, 53 mmol) was gradually added over 1 hour at 0° C. The mixture was washed with a 2M solution of hydrochloric acid in water (200 mL), and the organic phase was collected. The organic phase was purified via distillation to yield 2-chloro-2,3,3-trifluoro-3-(2,2,3,3,4,4,4-heptafluorobutoxy)propan-1-ol C₃F₇CH₂OCF₂CFClCH₂OH (18 g, 54% yield).

### Example 8

Example 8 illustrates the preparation of 2,2,3,3-tetrafluoro-3-(2,2,3,3,4,4,4-heptafluorobutoxy)propan-1-ol from carbonate. The 2,2,3,3,4,4,4-heptafluorobutyl methyl carbonate C₃F₇CH₂OC(O)OCH₃ was prepared as in Example 7.

Sodium hydride (60% in mineral oil, 2.4 g, 60 mmol) and anhydrous tetrahydrofuran (120 mL) were added to a 250-mL flask under nitrogen atmosphere. Then 2,2,3,3,4,4,4-heptafluorobutan-1-ol (10 g, 50 mmol) was slowly added. The mixture was stirred at 20 C.° for 15 minutes and then transferred to a 400-mL Hastelloy vessel, at which point the mixture was cooled to −30° C. and 2,2,3,3,4,4,4-heptafluorobutyl methyl carbonate (30 g, 116 mmol) was added to the vessel. The vessel was then pressurized with tetrafluoroethylene (20 g, 200 mmol), allowed to warm to ambient temperature, agitated for 3 hours, and then vented. The mixture was transferred to a 250-mL flask, cooled to 0° C., and lithium aluminum hydride (2.4 g, 63 mmol) was added. The mixture was stirred for 3 hours at a temperature between 5 and 20° C., and the resultant mixture was washed with a 1M solution of hydrochloric acid in water (200 mL). The organic phase was isolated and tetrahydrofuran was removed to vacuum. The remaining organic phase was purified via distillation to yield 2,2,3,3-tetrafluoro-3-(2,2,3,3,4,4,4-heptafluorobutoxy)propan-1-ol C₃F₇CH₂OCF₂CF₂CH₂OH (3 g, 8% yield).

### Example 9

Example 9 illustrates the preparation of 2,2,3,3,3-pentafluoropropyl formate and the subsequent preparation of 2-chloro-2,3,3-trifluoro-3-(2,2,3,3,3-pentafluoropropoxy)propan-1-ol. Formic acid (150 g, 3.26 mol) and 2,2,3,3,3-pentafluoropropan-1-ol (95 g, 633 mmol) were charged into a 500-mL flask, and the resultant mixture was refluxed at 80° C. for 2 hours. The crude 2,2,3,3,3-pentafluoropropyl formate was distilled off and collected, formic acid (70 g, 1.52 mol) was charged into the crude formate, and the distillation was repeated. The distillate was washed with water (50 mL) and dried over magnesium sulfate (MgSO₄) to afford pure 2,2,3,3,3-pentafluoropropyl formate C₂F₅CH₂OC(O)H (80 g, 71% yield).

Sodium hydride (60% in mineral oil, 1.2 g, 30 mmol) and anhydrous tetrahydrofuran (50 mL) were charged to a 250-mL flask under nitrogen atmosphere. Then 2,2,3,3,3-pentafluoropropan-1-ol (3.0 g, 20 mmol) was slowly added. The mixture was cooled to −20° C. and 2,2,3,3,3-pentafluoropropyl formate (5.0 g, 28 mmol) was added. Chlorotrifluoroethylene (5.0 g, 43 mmol) was gradually added by bubbling through the solution while maintaining the temperature between −20 and −30° C., and the mixture was stirred for 30 minutes at −20° C. Lithium aluminum hydride (0.7 g, 18 mmol) was added, and the mixture was stirred for an additional hour at −20° C. Then the mixture was allowed to warm up to room temperature and was washed with a 1M solution of hydrochloric acid in water (100 mL). The organic phase was distilled to yield 2-chloro-2,3,3-trifluoro-3-(2,2,3,3,3-pentafluoropropoxy)propan-1-ol C₂F₅CH₂OCF₂CFClCH₂OH (2 g, 24% yield).

### Example 10

Example 10 illustrates the preparation of 3-(2,2,3,3,4,4,4-heptafluorobutoxy)propan-1-ol from 2,2,3,3,4,4,4-heptafluorobutan-1-ol. Sodium hydride (60% in mineral oil, 5.0 g, 124 mmol) and anhydrous tetrahydrofuran (80 mL) were charged into a 250-mL flask, and 2,2,3,3,4,4,4-heptafluorobutan-1-ol (22.0 g, 110 mmol) was slowly added. Next, 3-bromopropan-1-ol (10.5 g, 76 mmol) was added, and the resultant mixture was heated at 50° C. for 3 hours. The reaction mixture was washed with 0.5M solution of aqueous HCl (100 mL), and the organic layer was isolated. The organic layer was distilled to yield 3-(2,2,3,3,4,4,4-heptafluorobutoxy)propan-1-ol C₃F₇CH₂OCH₂CH₂CH₂OH (10 g, or 51% yield).

### Example 11

Example 11 illustrates the preparation of 2-(2-(2,2,3,3,4,4,4-heptafluorobutoxy)ethoxy)ethanol from 2,2,3,3,4,4,4-heptafluorobutan-1-ol. Sodium hydride (60% in mineral oil, 6.0 g, 150 mmol) and anhydrous diglyme (80 mL) were charged into a 250-mL flask, and 2,2,3,3,4,4,4-heptafluorobutan-1-ol (30.0 g, 150 mmol) was slowly added. Next, 2-(2-chloroethoxy)ethanol (12.4 g, 100 mmol) was added, and the resultant mixture was heated at 110° C. for 2 hours.

The reaction mixture was washed 0.5M solution of aqueous HCl (100 mL), and the organic layer was isolated. The organic layer was distilled to yield 2-(2-(2,2,3,3,4,4,4-heptafluorobutoxy)ethoxy)ethanol C₃F₇CH₂OCH₂CH₂OCH₂CH₂OH (10 g, 35% yield).

### Example 12

Example 12 illustrates the preparation of 6-(2,2,3,3,4,4,4-heptafluorobutoxy)hexan-1-ol from 2,2,3,3,4,4,4-heptafluorobutan-1-ol. Sodium hydride (60% in mineral oil, 6.0 g, 150 mmol) and anhydrous monoglyme (100 mL) were charged into a 250-mL flask, and 2,2,3,3,4,4,4-heptafluorobutan-1-ol (30.0 g, 150 mmol) was slowly added. Next, 6-bromohexan-1-ol (21.0 g, 116 mmol) was added, and the resultant mixture was heated at reflux for 4 hours. The reaction mixture was washed with a 1M solution of aqueous HCl (100 mL) and the organic layer was isolated. The organic layer was distilled to yield 6-(2,2,3,3,4,4,4-heptafluorobutoxy)hexan-1-ol C₃F₇CH₂O(CH₂)₆OH (20.6 g, 59% yield).

### Example 13

Example 13 illustrates the preparation of 8-(2,2,3,3,4,4,4-heptafluorobutoxy)octan-1-ol from 2,2,3,3,4,4,4-heptafluorobutan-1-ol. Cesium carbonate (13 g, 40 mmol) and anhydrous diglyme (80 mL) were charged into a 250-mL flask, and 2,2,3,3,4,4,4-heptafluorobutan-1-ol (16.0 g, 80 mmol) was slowly added. The resultant mixture was heated to 125° C. Then, 8-chlorooctan-1-ol (12 g, 73 mmol) was added, and the mixture was stirred at 125° C. After 4 hours, additional cesium carbonate (13 g, 40 mmol) was added, and heating resumed for an additional 20 hours at 125° C. After that, the reaction mixture was filtered and washed with 0.5M solution of aqueous HCl (100 mL) and the organic layer was isolated. The organic material was distilled to yield 8-(2,2,3,3,4,4,4-heptafluorobutoxy)octan-1-ol C₃F₇CH₂O(CH₂)₈OH (8.4 g, 29% yield).

### Example 14

Example 14 illustrates the preparation of 3-(2,2,3,3-tetrafluoro-3-(2,2,3,3,3-pentafluoropropoxy)propoxy)-propan-1-ol from the alcohol in Example 5. Sodium hydride (60% in mineral oil, 4.4 g, 110 mmol) and anhydrous tetrahydrofuran (80 mL) were charged into a 250-mL flask, and 2,2,3,3-tetrafluoro-3-(2,2,3,3,3-pentafluoropropoxy)propan-1-ol (18.0 g, 64 mmol) was slowly added. Then, 3-bromopropan-1-ol (8.0 g, 58 mmol) was added, and the resultant mixture was heated at 60° C. for 4 hours. The mixture was washed with 0.5M solution of aqueous HCl (100 mL), and the organic layer was isolated. The organic material was distilled to yield 3-(2,2,3,3-tetrafluoro-3-(2,2,3,3,3-pentafluoropropoxy)propoxy)-propan-1-ol C₂F₅CH₂OCF₂CF₂CH₂O(CH₂)₃OH (8.5 g, 43% yield).

### Example 15

Example 15 illustrates the preparation of 2-(2,2,3,3-tetrafluoro-3-(2,2,3,3,3-pentafluoropropoxy)propoxy)-ethanol from the alcohol in Example 5. Boric acid (0.3 g, 5 mmol) and 2,2,3,3-tetrafluoro-3-(2,2,3,3,3-pentafluoropropoxy)propan-1-ol (31.4 g, 112 mmol) were charged into a 100-mL flask equipped with a 10-mL Dean-Stark condenser, and the mixture was heated at 125° C. for 1 hour. The mixture was then transferred into a Hastelloy vessel (400 mL), and tetrabutylammonium iodide (1.5 g, 4 mmol) was added. The vessel was pressurized with ethylene oxide (5 g, 114 mmol) and heated at 110° C. for 6 hours. At this point, the mixture was transferred to a 100-mL flask, a solution of sodium hydroxide (3.0 g, 75 mmol) in water (30 mL) was added, and the mixture was stirred at 70° C. for 20 minutes. The mixture was cooled, the organic phase was separated, and the product was distilled to yield 2-(2,2,3,3-tetrafluoro-3-(2,2,3,3,3-pentafluoropropoxy)propoxy)-ethanol C₂F₅CH₂OCF₂CF₂CH₂OCH₂CH₂OH (4.4 g, 19% yield).

### Example 16

Example 16 illustrates the preparation of 3-(2,2,3,3-tetrafluoro-3-(2,2,3,3,4,4,4-heptafluorobutoxy)propoxy)-propan-1-ol from the alcohol in Example 6. Sodium hydride (60% in mineral oil, 3.2 g, 80 mmol) and anhydrous tetrahydrofuran (80 mL) were charged into a 250-mL flask, and 2,2,3,3-tetrafluoro-3-(2,2,3,3,4,4,4-heptafluorobutoxy)propan-1-ol (25 g, 76 mmol) was slowly added. The resultant mixture was stirred for 10 minutes, and 3-bromopropan-1-ol (9.5 g, 68 mmol) was added. The resultant mixture was heated at 50° C. for 8 hours, then washed with 0.5M solution of aqueous HCl (100 mL). The organic layer was isolated and distilled to yield 3-(2,2,3,3-tetrafluoro-3-(2,2,3,3,4,4,4-heptafluorobutoxy)propoxy)-propan-1-ol C₃F₇CH₂OCF₂CF₂CH₂O(CH₂)₃OH (12.4 g, 47% yield).

### Example 17

Example 17 illustrates the preparation of 3-(2,2,3,3,4,4,4-heptafluorobutoxy)propyl 3-mercaptopropanoate from the alcohol in Example 10. Cyclohexane (˜70 mL), 3-(2,2,3,3,4,4,4-heptafluorobutoxy)propan-1-ol (10 g, 38.7 mmol), 3-mercaptopropanoic acid (4.3 g, 40.5 mmol), and toluenesulfuric acid (0.2 g) were charged into a 250-mL flask equipped with a Dean-Stark condenser, and the resultant mixture was heated at reflux for 3 hours. The mixture was washed with water (100 mL), the organic phase was isolated, and cyclohexane was removed under reduced pressure. The organic material was distilled to yield 3-(2,2,3,3,4,4,4-heptafluorobutoxy)propyl 3-mercaptopropanoate C₃F₇CH₂O(CH₂)₃OC(O)CH₂CH₂SH (11.4 g, 85% yield).

### Example 18

Example 18 illustrates the preparation of 6-(2,2,3,3,4,4,4-heptafluorobutoxy)hexyl 3-mercaptopropanoate from the alcohol in Example 12. The procedure of Example 17 was performed, except that 6-(2,2,3,3,4,4,4-heptafluorobutoxy)hexan-1-ol (11.2 g, 37 mmol), 3-mercaptopropanoic acid (5.0 g, 47 mmol), and toluenesulfuric acid (0.2 g) were reacted. Pure 6-(2,2,3,3,4,4,4-heptafluorobutoxy)hexyl 3-mercaptopropanoate C₃F₇CH₂O(CH₂)₆OC(O)CH₂CH₂SH was isolated (11 g, 76% yield).

### Example 19

Example 19 illustrates the preparation of 8-(2,2,3,3,4,4,4-heptafluorobutoxy)octyl 3-mercaptopropanoate from the alcohol in Example 13. The procedure of Example 17 was performed, except that 8-(2,2,3,3,4,4,4-heptafluorobutoxy)octan-1-ol (8.4 g, 25.6 mmol), 3-mercaptopropanoic acid (3.2 g, 30 mmol), and toluenesulfuric acid (0.2 g) were reacted. Pure 8-(2,2,3,3,4,4,4-heptafluorobutoxy)octyl 3-mercaptopropanoate C₃F₇CH₂O(CH₂)₈OC(O)CH₂CH₂SH was isolated (7.3 g, 69% yield).

### Example 20

Example 20 illustrates the preparation of 3-(2,2,3,3-tetrafluoro-3-(2,2,3,3,3-pentafluoropropoxy)propoxy)propyl 3-mercaptopropanoate from the alcohol in Example 14. The procedure of Example 17 was performed, except that 3-(2,2,3,3-tetrafluoro-3-(2,2,3,3,3-pentafluoropropoxy)propoxy)propan-1-ol (8.6 g, 25.4 mmol), 3-mercaptopropanoic acid (2.8 g, 26.4 mmol), and toluenesulfuric acid (0.1 g) were reacted. Pure 3-(2,2,3,3-tetrafluoro-3-(2,2,3,3,3-pentafluoropropoxy)propoxy)propyl 3-mercaptopropanoate C₂F₅CH₂OCF₂CF₂CH₂O(CH₂)₃OC(O)CH₂CH₂SH was isolated (7.1 g, 65% yield).

### Example 21

Example 21 illustrates the preparation of 3-(2,2,3,3,4,4,4-heptafluorobutoxy)propyl methacrylate from the alcohol in Example 10. Cyclohexane (50 mL), 3-(2,2,3,3,4,4,4-heptafluorobutoxy)propan-1-ol (21 g, 81 mmol), p-toluenesulfonic acid (1.0 g, 5 mmol), and 4-methoxyphenol (0.08 g, 0.6 mmol) were added to a 250-mL flask equipped with a Dean-Stark trap. The resultant mixture was sparged with air for 5 minutes, and then methacrylic acid (9.8 g, 114 mmol) was added. The mixture was refluxed for 12 hours, cooled down, washed with water (2×50 mL), and the organic layer was separated. The organic material was distilled to yield 3-(2,2,3,3,4,4,4-heptafluorobutoxy)propyl methacrylate C₃F₇CH₂O(CH₂)₃OC(O)C(CH₃)═CH₂ (22.7 g, 85% yield).

### Example 22

Example 22 illustrates the preparation of 3-(2,2,3,3-tetrafluoro-3-(2,2,3,3,3-pentafluoropropoxy)propoxy)propyl methacrylate from the alcohol in Example 14. The procedure of Example 25 was performed, except that 3-(2,2,3,3-tetrafluoro-3-(2,2,3,3,3-pentafluoropropoxy)propoxy)-propan-1-ol (21.6 g, 64 mmol), p-toluenesulfonic acid (1.0 g, 5 mmol), 4-methoxyphenol (0.08 g, 0.6 mmol), and methacrylic acid (7.7 g, 89 mmol) were reacted. The mixture was refluxed for 14 hours, cooled down, washed with water (2×50 mL), and the organic layer was separated. Pure 3-(2,2,3,3-tetrafluoro-3-(2,2,3,3,3-pentafluoropropoxy)propoxy)propyl methacrylate C₂F₅CH₂OCF₂CF₂CH₂O(CH₂)₃OC(O)C(CH₃)═CH₂ was isolated (20.5 g, 79% yield).

What is claimed is:

1. A compound of formula (1): 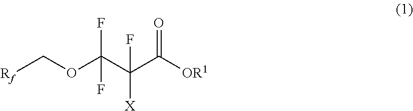
wherein R_{f} is C₁ to C₆ linear or branched fluoroalkyl, X is F or Cl, and R¹ is H, CH₃, or CH₂CH₃.

2. The compound of claim 1 wherein R_{f} is C₁ to C₃ linear fluoroalkyl.

3. A process to prepare a compound of Formula (1) of claim 1 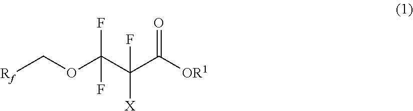
wherein R_{f} is C₁ to C₆ linear or branched fluoroalkyl and X is F or Cl, comprising
a) contacting a alkali metal hydride with a fluorinated alcohol of formula R_{f}CH₂OH to produce a catalyst, b) contacting a symmetrical fluorinated carbonate of formula (R_{f}CH₂O)₂C(O), wherein R_{f} is C₁ to C₆ linear or branched fluoroalkyl, with CF₂═CFX, wherein X is F or Cl, in the presence of the catalyst to generate a reaction mixture, c) hydrolyzing the reaction mixture of step b) to form a compound of Formula (1a), 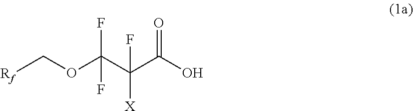
and d) optionally reacting the compound of Formula (1a) with methanol or ethanol to yield a compound of Formula 1.

4. The process of claim 3 wherein R_{f} is C₁ to C₃ linear fluoroalkyl.

5. A compound of formula (2): 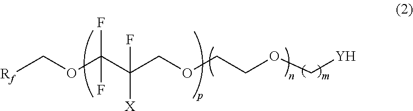
wherein
R_{f} is C₁ to C₆ linear or branched fluoroalkyl,
X is F or Cl,
Y is O or a single bond,
p is 0 to 1,
m is 0 or 3 to 10, and
n is 0 to 30,
wherein at least one of p or m is a positive integer;
provided that, if n is a positive integer, then p is 1; and
provided that, if m is 0 then Y is a single bond, and if m is a positive integer then
Y is O.

6. The compound of claim 5, wherein R_{f} is C₁ to C₃ linear fluoroalkyl.

7. The compound of claim 5 selected from the formula (2) such that (1) p is 1, m is 0 or 3 to 8, and n is 0; (2) p is 0, m is 3 to 8, and n is 0; or (3) p is 1, m is 0, and n is 1 to 12.

8. A process to prepare a compound of formula (2) of claim 5 comprising
a) contacting an alkali metal hydride of formula WH_{g}, where W is an alkali metal and g is 1 to 2, with a fluorinated alcohol of formula R_{f}CH₂OH, wherein R_{f} is C₁ to C₆ linear or branched fluoroalkyl, to produce a catalyst of formula R_{f}CH₂O⁻W⁺,
b) contacting R_{f}CH₂OH with (i) formic acid to produce a partially fluorinated formate, (ii) alkyl chloroformate to produce a mixed partially fluorinated carbonate, or (iii) phosgene, diphosgene, or triphosgene to produce a symmetrical partially fluorinated carbonate,
c) contacting the partially fluorinated formate of step (b)(i) or partially fluorinated carbonate of step (b) (ii) or (b) (iii) with CF₂═CFX, wherein X is F or Cl, in the presence of the catalyst of formula R_{f}CH₂O⁻W⁺ to yield, respectively, a partially fluorinated aldehyde (i), partially fluorinated ester (ii), or partially fluorinated carboxylic acid (iii),
d) contacting the partially fluorinated carboxylic acid of step (c) (iii) with an organic alcohol to form a partially fluorinated ester,
e) contacting (i) the partially fluorinated aldehyde of step (c) (i), (ii) the partially fluorinated ester of step (c) (ii), or (iii) the partially fluorinated ester of step (d) with a reducing agent to form the compound of formula (2a), and 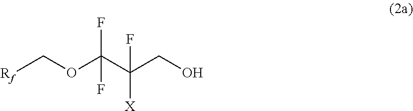
f) optionally contacting the compound of formula (2a) with an alcohol of the formula J(CH₂)ₜOH in the presence of a base, where J is a halogen and t is 3-10, or ethylene oxide in the presence of a catalyst, to form the compound of formula (2).

9. A process to prepare a compound of formula (2) of claim 5 comprising contacting an alcohol of the formula R_{f}CH₂OH, wherein R_{f} is C₁ to C₆ linear or branched fluoroalkyl, with an alcohol of formula J(CH₂)ₜOH, where J is a halogen and t is 3-10, in the presence of a base.

10. The process of claim 9, wherein the base is chosen from NaH, KOH, NaOH, or Cs₂CO₃.

11. The process of claim 8 or 9, wherein R_{f} is C₁ to C₃ linear fluoroalkyl.

12. The process of claim 8, wherein steps c) and e) are performed as a semi-continuous process where the partially fluorinated aldehyde of step (c) (i) or partially fluorinated ester of step (c) (ii) is not isolated prior to performing step e).

13. The process of claim 8, wherein the partially fluorinated formate of step (b) (ii) is formed without the use of organic solvent.

14. The process of claim 8, where the alkali metal hydride of step a) is selected from the group consisting of NaH, KH, and CaH₂; and the reducing agent of step d) is selected from the group consisting of LiAlH₄ and NaBH₄.

15. The process of claim 8, wherein steps (c) (i), (c) (ii), (e) (i), and (e) (ii) take place at a temperature at or below −20° C.

16. A compound of Formula (3): 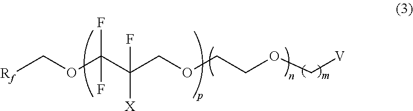
wherein
R_{f} is C₁ to C₆ linear or branched fluoroalkyl,
X is F or Cl,
V is —YC(O)CR²═CH₂, —YC(O)R¹⁹SH, or —YC(O)NHCH₂CH₂OC(O)CR²═CH₂,
Y is O or a single bond,
R² is independently selected from H or a C₁ to C₄ alkyl group,
R¹⁹ is an alkylene of about 1 to about 10 carbon atoms,
p is 0 to 1,
m is 0 or 3 to 10, and
n is 0 to 30,
wherein at least one of p or m is a positive integer;
provided that, if n is a positive integer, then p is 1; and
provided that, if m is 0 then Y is a single bond, and if m is a positive integer then
Y is O.

17. The compound of claim 19, wherein R_{f} is C₁ to C₃ linear fluoroalkyl.
